(19) United States
(12) Patent Application Publication
(10) Pub. No.: US 2004/0068100 A1
(43) Pub. Date: Apr. 8, 2004

(54) Multicomponent conjugates which bind to target molecules and stimulate cell lysis

(76) Inventors: Jean-Pierre Mach, CH-1293 Bellevue (CH); Bruno Robert, Saint Hippocyte Du Fort (FR); Pedro Romero, Epalinges (CH); Immanuel Leuscher, CH-1124 (CH); Philipped Guillaume, Lausanne (CH)

Correspondence Address: FULBRIGHT & JAWORSKI, LLP, 666 FIFTH AVE, NEW YORK, NY, 10103-3198, US

(21) Appl. No.: 10/276,764

(22) PCT Filed: May 24, 2001
(86) PCT No.: PCT/US01/17184

(51) Int. Cl. C07K 16/46; C07K 16/28

(52) U.S. Cl. 530/388.22; 530/391.1

(57) ABSTRACT

The invention relates to immunoconjugates of formula: A—B—(C)n where B may be present or absent, A is a specific binding protein such as an antibody or an antibody binding fragment, or a ligand binding to a receptor present on target cells, B comprises at least one molecule to which “A” and “C” bind, such as an avidin/strepavidin complex, “C” is an MHC molecule, and “n” is a whole number ranging from 1 to 10. The conjugates provide the exquisite binding specificity of antibodies, combined with an ability to stimulate cytotoxic T cells to identify and to destroy cells on which the conjugate is bound and oligomerized. The conjugates are useful both therapeutically and diagnostically.

### FIELD OF THE INVENTION

This invention relates to conjugates, or fusion proteins which comprise a specific, cell surface binding molecule, and an antigenic complex of an MHC molecule and a peptide. Such constructs bind to target cells, leading to activation of T lymphocytes, and induction of cytotoxicity.

### BACKGROUND AND PRIOR ART

Antibodies are high molecular weight proteins which recognize and bind specifically to molecules, such as foreign molecules (e.g., proteins, glycoproteins, lipoproteins, etc.), which are sometimes referred to as antigens, or markers. The term “marker” is used frequently when the antibody target is found on the surface of a subpopulation of cells, such as tumor cells, or cells bearing one or more differentiation antigens, also called “clusters of differentiation” or “CDs.” Antibodies bind to specific epitopes formed by the target molecule. While antibodies are known for their excellent binding and targeting ability, they are not particularly efficient at killing target cells to which they bind.

Antibodies represent just one facet of the immune system. T lymphocytes are cells which have surface receptors that are capable of recognizing, e.g., viral or tumor antigens, only in the form of short peptides, presented within the groove of so-called “major histocompatibility complexes” or “MHC”s on the surface of cells. The recognition of and binding to peptides associated with MHC on the surface of target cells leads to the activation of specific T lymphocytes, and often to the lysis of the cells expressing the specific MHC peptide complex. This is a very efficient killing mechanism; however, sometimes virally infected cells or, more frequently, tumor cells, escape the T lymphocyte attack by, e.g., deleting expression of molecules that are a part of the MHCs, and hence their expression on cell surfaces.

There are two classes of MHC molecules, i.e., “Class I” and “Class II” MHC molecules. The first class is expressed on the surface of most human cells, while constitutive expression of Class II molecules is limited for the most part to B lymphocytes, dendritic cells, and macrophages. These three cell types function as “antigen presenting cells.”

Structurally, MHC Class I molecules consist of three components: (i) a heavy chain with a molecular weight of about 50 kilodaltons, (ii) a light, non-polymorphic chain, referred to as beta-2microglobulin, “beta2M”, or “β2M” and (iii) a peptide which generally consists of 8-10 amino acids which lies in a specific groove made by the heavy chain N terminal domain of MHC. The first item, i.e., the heavy chain, exhibits genetic polymorphism at its extracellular N-terminus, and a non-polymorphic, partially intracellular C-terminus. The third item, i.e., the peptide, varies, depending upon the nature of the polymorphism in (i). When these three elements form an MHC presented on cell surfaces, the complex is referred to as a T cell antigen, and CD8⁺ T lymphocytes with appropriate receptors bind to them and act as described supra. See Townsend, et al., Ann. Rev. Immunol 7:601-24 (1989), incorporated by reference, for a discussion of this structure.

The MHC Class II molecules which consist of two chains, α and β, of similar size, present longer peptides, 15-25 amino acids long, to CD4+ T lymphocytes. Soluble, recombinant Class I and II MHCs have been expressed in bacteria and insect cells, respectively (Garboczi, et al, Proc. Natl. Acad Sci USA 89:3429-33 (1992), Stern, et al, Nature 368:215-21 (1994) incorporated by reference). Further, artificial forms of recombinant MHC Class I have been synthesized, which consist of a single chain containing all three of the aforementioned elements. These molecules were synthesized via using genes encoding a fusion protein. These molecules retained their capacity to be recognized by T lymphocytes. See Mottez, et al, J. Exp. Med 181:493-502 (1995), incorporated by reference.

Recently, recombinant MHC Class I molecules which contain 15 amino acid sequences at their C terminus that allow the site specific coupling of biotin on a lysine residue by the BirA enzyme, have been synthesized. See Schatz, et al, Biotechnology 11:1138-43 (1993) (1996), incorporated by reference. These molecules can be biotinylated at their C-terminal end, which permits tetramerization via binding of biotin molecules to the four binding sites on avidin or streptavidin. These tetramers bind with higher affinity to T lymphocytes expressing specific receptors, thanks to multiple cooperative bonds. See Altman, et al, Science 274:94-6 (1996) incorporated by reference. In addition, if the streptavidin or avidin molecule used is labelled with, e.g., a fluorescent molecule, such as phycoerythrin, the tetramers can be used in vitro in order to characterize T cells specific for a given antigenic peptide via, e.g., flow cytofluorimetry. These complexes have been used to characterize the afferent arm of the T lymphocyte response, but not for the study of their effector properties.

One aspect of the invention relates to conjugates which combine the high binding specificity of specific, cell surface binding molecules, such as antibodies for their targets, or ligands for various receptors, and the capacity of MHC/peptide complexes, oligomerized on target cells, to stimulate specific cytolytic T lymphocytes.

It is a further aspect of the invention to present conjugates of Fab′ fragments, MHC molecules and peptides, which provide a real link between the antigen recognition property of antibodies which bind to epitopes on large, native molecules, and the recognition properties of T cell receptors, which bind to antigenic, short peptides expressed in MHC complexes.

It is a further aspect of the invention to provide a method for eliminating target cells by contacting these with conjugates of the type described supra and inducing their lysis by T lymphocytes.

It is a further aspect of the invention to provide conjugates which include the specific binding proteins coupled to MHC, described supra is directed against an antigen or marker expressed on an antigen presenting cell. When oligomerized on the surface of antigen presenting cells, the MHC/peptide complexes stimulate T cells, in a manner mimicking the vaccination effect.

Multimeric complexes of streptavidin, biotin, and MHC molecules are known from the art. See, in this regard, Dunbar, et al, Tumor Immunol 92(12):3.3 (1997); Altman et al, supra, and PCT publication WO 99/50637, to Romero, et al; all of which are incorporated by reference. Apart from describing the general concept of streptavidin-biotin-MHC multimeric complexes, the PCT publication describes how these can be adapted to the class of molecules referred to as tumor rejection antigens, or “TRAs.” More information on TRAs can be found in, e.g., U.S. Pat. Nos. 6,025,470; 5,554,724; 5,554,506, and 5,487,974, all of which are incorporated by reference. The concept of the tumor rejection antigen is described in U.S. Pat. No. 5,342,774, also incorporated by reference. There is a vast patent literature on these molecules, and the specific members of the family of MHC molecules of which they are a part.

None of these references suggest, however, that the monomeric MHC/peptide complexes could be directly conjugated or fused to binding proteins, such as antibodies or binding fragments of antibodies directed against markers abundantly expressed on the surface of target cells, can induce the oligomerization of MHC complexes on target cells resulting in optimal recognition by T lymphocytes. Such bifunctional conjugates, which are described herein, are useful in targeting specific cells, as well as in destroying these targeted cells, via the intervention of cytolytic T cells.

Furthermore, when the target cells of such conjugates containing a binding protein and MHC class I or class II MHC/peptide complexes belong to the category of antigen presenting cells, such as dendritic cells or B lymphocytes, activation of specific CD8 or CD4 T lymphocytes can be induced.

### BRIEF DESCRIPTION OF THE FIGURES

FIG. 1 presents flow cytometry analysis demonstrating the specific coating of anti-tumor Fab-HLA-A2/flu conjugates on the surface of HLA-A2 negative tumor cells, using a FITC-labeled anti-HLA-A2 mAb.

FIGS. 2A-2C shows results of induction of specific lysis, measured by a ⁵¹Cr release assay, of different types of cancer cells preincubated with a constant amount of 2 μg/ml of said bifunctional conjugates in accordance with the invention, with a titration of specific cytotoxic T lymphocyte at effector to target cell ratio, ranging from 0.1 to 30/1. FIG. 2A shows results using anti-CEA Fab′ conjugates, 2B shows results obtained using anti-HER2 Fab′ containing conjugates, and 2C shows results using anti-CD20 Fab′ conjugates.

FIGS. 3A-C shows results of induction of specific lysis of different types of cancer cells preincubated with different amounts of said bifunctional conjugates in accordance with the invention, ranging from 10⁻¹ to 10³ ng/ml in presence of a constant effector to target cell ratio of 10/1. The panels parallel those of FIG. 2.

FIG. 4 schematically describes the mechanism, by which the described Fab-MHC conjugates can induce very efficient target cell killing by specific cytotoxic T lymphocytes, through oligomerization of the conjugate on the target cell surface.

### DETAILED DESCRIPTION OF PREFERRED EMBODIMENTS

### EXAMPLE 1

As pointed out, supra, soluble MHC molecules are known to the art. A nucleic acid molecule encoding soluble HLA-A*0201 heavy chain was treated to introduce two site directed mutations. Specifically, the codon for glutamic acid at position 275 was mutated to free cysteine, and a stop codon was introduced at position 279, on the C terminal portion of the 3d domain of the heavy chain. Commercially available products, and art recognized methodologies were used in this step. Briefly, however, the expression plasmid pHN1 HLA-A2-BSP, taught by Altman, et al, Science 274:94-6 (1996), incorporated by reference, was used, in combination with polymerase chain reaction and sequencing methodologies to introduce and to confirm the mutations.

The mutated molecule was then used, together with a nucleic acid molecule encoding β2M, in accordance with Altman, et al, supra, Romero, et al, J. Exp. Med 188:1641-1650 (1998), and/or Garboczi, et al, Proc. Natl. Acad. Sci USA 89:3429-3433 (1992) all of which are incorporated by reference. The expression of the proteins was carried out in E. coli, resulting in inclusion bodies.

The inclusion bodies were refolded, and combined with the known HLA-A2 restricted, immunodominant influenza virus Flu matrix peptide “FLUMA 58-66”, i.e.:

Gly Ile Leu Gly Phe Val Phe Thr Leu (SEQ ID NO:1),

resulting in MHC peptide complexes which were purified on a column.

These monomeric MHC conjugates were used in the further examples which follow.

### EXAMPLE 2

This example describes the formation of conjugates consisting of a monomeric MHC peptide complex of example 1, and a single murine Fab′ fragment specific to carcinoembryonic antigen, or “CEA.”

Buchegger, et al, J. Exp. Med 158:413-427 (1983), incorporated by reference, describe murine IgG1 monoclonal antibody 35A7, against CEA. The mAb displays no cross reactivity for antigens expressed by granulocytes.

Monoclonal antibodies were incubated with pepsin, at a 3:100 wt/wt ratio of pepsin/mAb, and incubated at 37° C. in 0.2M acetate buffer, pH 4.0, for 22 hours, to produce F(ab′)₂ fragments. In turn, the F(ab′)₂ fragments were reduced with 10 mM cysteamine, for 1 hour at 37° C., in Hepes/NaCl buffer, pH 7.0, and then separated on a column. This yielded the Fab′ fragments.

In order to conjugate the fragments with the molecules of example 1, the latter were incubated for 2 hours with a 25 molar excess of bismaleimide polyethylene oxide at room temperature, in phosphate buffered saline, pH 7.0. The excess coupling reagent was removed via gel filtration, resulting in 45 kilodalton, bismaleimide derivatized MHC molecules, containing a free thiol group at position 275.

These derivatives were combined, immediately, with a 1.5 molar excess of Fab′ fragments, freshly prepared as described in this example, followed by 18 hours of incubation at 4° C., after concentration of the proteins to 10 mg/ml. Conjugates were purified via FPLC, using commercially available products and known methods, and then analyzed under both reducing and nonreducing conditions, using 10% SDS-PAGE gel electrophoresis.

The conjugates eluted at an apparent molecular weight of about 95 kilodaltons from a molecular sieving column equilibriated in non-denaturing buffer. They showed a major band of about 82 kilodaltons on SDS-PAGE, under non-reducing conditions, apparently due to dissociation of β2M and peptide.

Under reducing conditions, a single band of about 57 kilodaltons was obtained, corresponding to a thioether linked HLA-A2 heavy chain, and a pepsin cleaved, Fab′ heavy chain. The same conjugate could be obtained by derivatizing the mAb with bismaleimide, followed by coupling to the monomeric MHC complex of examples linked HLA-A2 heavy chain and pepsin cleaved Fab′ heavy chain.

### EXAMPLE 3

This example describes the preparation of additional conjugates. Commercially available antibodies were used. Specifically, HERCEPTIN® is a recombinant, humanized mAb, of human IgG1κ isotype, specific for the extracellular domain of the HER2 receptor. See Carter, et al, Proc. Natl. Acad. Sci USA 89:4285-9 (1992), incorporated by reference. RITUXIMAB® is a chimeric, murine/human mAb of IgG1 human κ subtype, directed against the CD20 molecule found on the surfaces of normal and malignant B lymphocytes. See Reff, et al, Blood 83:435-445 (1994), incorporated by reference.

The same protocol that was used to prepare Fab′ fragments in example 2 was used, with the following exceptions: The HERCEPTIN F(ab′)₂ fragments were incubated with pepsin for 8 hours, and RITUXIMAB was incubated for 15 hours.

Fab′ fragments, and conjugates with monomeric MHC molecules were prepared exactly as described in example 2.

### EXAMPLE 4

This example describes flow cytometry analyses of the conjugates described in examples 2 and 3, supra.

Various cell lines were used, including LoVo, which is a colon carcinoma cell line that expresses CEA, SK-BR-3, which is a breast carcinoma cell line expressing HER 2 (ErbB2), and B cell lymphomas Daudi and Raji, both of which express CD20. The cells are all commercially available from the American Type Culture Collection. They were cultured in RPMI 1640, supplemented with 10% fetal calf serum. Daudi cells express no MHC Class I molecules, due to deletion of the β2M gene. The other three cell lines are known to be HLA-A2 negative, a fact which was confirmed via assaying with an HLA-A2 specific antibody.

Samples of LoVo, SK-BR3 and Daudi cell lines were incubated with each conjugate, in 50 μl of PBS, containing 2% BSA, at a concentration of 2 μg/ml for 1 hour at room temperature under gentle agitation. Cells were washed, three times, and then FITC labelled, anti HLA-A2 mAbs were added, and incubated for 30 minutes at 4° C. The cells were washed, twice, and analyzed immediately via FACS. As negative controls, cells which were not incubated with conjugate were used.

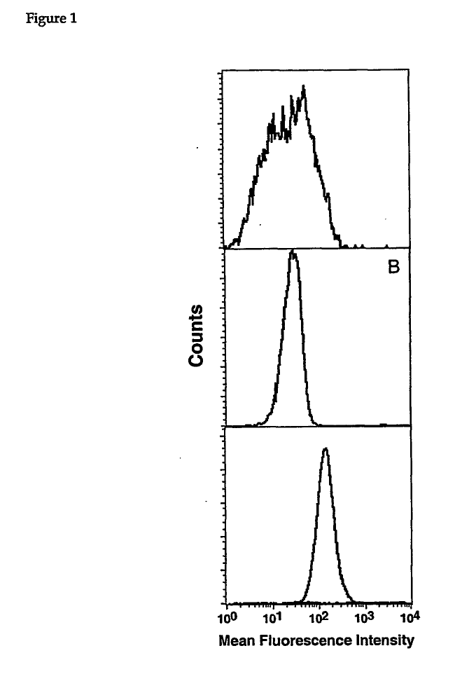

The results as set forth in FIG. 1 showed that, after incubation with the relevant bifunctional conjugates, all three cell lines presented a high density of HLA-A2, indicating that the antibody fragment portion of the bifunctional conjugates had specifically bound to the cell surfaces, and had specifically coated the monomeric MHC/peptide complexes.

### EXAMPLE 5

These experiments were carried out to determine if CTLs specific for the MHC/peptide complexes would recognize the tumor cells coated with bifunctional conjugates containing a monomeric MHC/peptide complex. Samples of each of the 4 cell lines were incubated for 45 minutes at 37° C., with a 2 μg/ml concentration of monomeric conjugate. The cells were labeled, concurrently, with ⁵¹Cr. Following labelling, cells were washed, three times, with PBS-2% BSA, as described supra, and then 1000 cell samples were incubated, at 37° C. for 4 hours, with an HLA-A2 restricted CTL clone specific for the SEQ ID NO: 1/HLA-A2 complex, as described by Valmori, et al, Canc. Res 59:4050-5 (1999), incorporated by reference. Varying effector: target ratios were used, i.e., 0.1, 1, 10 and 30:1. The cells were incubated in 200 μDMEM, 10% FCS, in V-bottomed microwell plates. Release of radiolabelled chromium was determined in accordance with Valmori, et al, J. Immunol 160:1750-8 (1998), incorporated by reference.

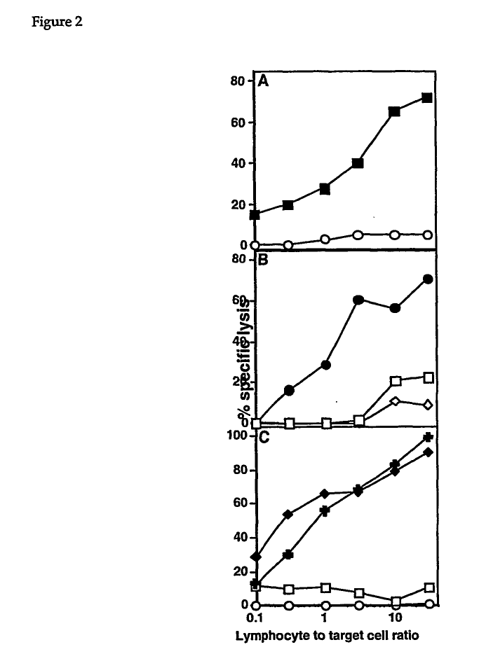

In a first experiment, LoVo cells, which express CEA, were lysed very efficiently by specific CTLs when preincubated with the conjugate containing anti-CEA Fab′ (filled square, panel A), while SK-BR-3 cells, which do not express the molecule, were not (open circles, panel A). These SK-BR-3 cells do express HER2, and were lysed after incubation with the conjugate containing anti-HER2 Fab′ (panel B, filled circles), with minimal lysis of LoVo cells (panel B, open squares) and almost no lysis of Daudi cells (panel B, open diamonds). Daudi and Raji cells both express CD20, and were lysed when incubated the conjugate containing the Fab′ fragment from the CD20 specific mAb (panel C, filled diamonds and crosses), while LoVo and SK-BR-3, which do not express the CD 20, were not (panel C, open squares and circles). In these experiments, the target cells were preincubated with a constant amount of conjugate (2 μg/ml) and the effector to target cell ratio ranged from 1:1 to 30:1; All of these results are set forth in FIGS. 2A-2C.

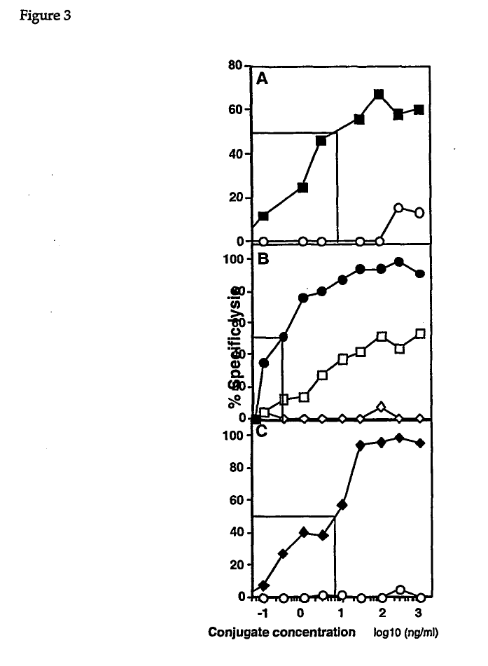

These experiments were continued, in order to titrate the conjugates for determining induction of CTL mediated tumor cell lysis at constant effector/target ratios of 10:1. Increasing concentrations, ranging from 10⁻¹ to 10³ ng/ml of conjugate, were incubated with the different cell lines described supra, for one hour, at 37° C., after which CTLs were added, and incubated for 4 hours, after which ⁵¹Cr release was measured. The anti-CEA conjugates were incubated with LoVo cells (filled squares), or SK-BR-3 (open circles), in panel A of FIG. 3. In panel B, anti-HER2 conjugates were incubated with SK-BR-3 (filled circles), LoVo cells (open squares), or Daudi cells (open diamonds). In panel C, the anti-CD20 conjugate was used with Daudi cells (filled diamonds), or SK-BR-3 (open circles). In each panel, the conjugate concentration giving 50% specific lysis is indicated. FIG. 3 shows that the conjugate concentration required for 50% lysis ranges from 0.5-8 ng/ml, 5-100 picomolar.

There was one instance where non-specific CTL mediated lysis appeared to occur. LoVo cells express barely detectable levels of HER2, but there was a moderate degree of lysis observed. See FIGS. 2B and 3B, open squares. As such, further experiments were carried out. In these experiments, whole monoclonal antibodies against HER2 (i.e., “HERCEPTIN”) were added, at 20 μg/ml, or not, with increasing concentrations of the anti-HER2 conjugates, to either SK-BR-3 or LoVo cells, and the CTLs described supra.

Unconjugated whole mAb to HER2 inhibited lysis of both SK-BR-3 and LoVo cells to the same degree, confirming that the lysis was due to the specificity of the antibody fragment of the conjugate indicating that the moderate degree of lysis was specific, probably due to low expression of HER-2 on LoVo cells. What was also observed was that, notwithstanding 20 μg/ml of competing mAb, the conjugates were still able to stimulate maximal lysis of SK-BR-3 at concentrations of 1 μg/ml or higher, confirming the high potency of the conjugate.

The results demonstrate that a bifunctional conjugate containing a monomeric form of MHC viral peptide complex and a single monovalent anti-tumor marker antibody fragment can induce very efficient and sensitive lysis of epithelial and lymphoid cancer cells by viral specific cytotoxic T lymphocytes. Cells coated by the antibody Fab′ fragment, monomeric MHC/viral peptide complexes are lyzed as efficiently as if they were infected by the relevant virus.

### EXAMPLE 6

These experiments were designed to determine the ability of the conjugates described supra, to mobilize intracellular Ca²⁺ in specific CTLs. See Valitutti, et al, J. Exp. Med 181:577-584 (1995), incorporated by reference for a discussion of the phenomenon of Ca²⁺ mobilization in CTLs following T cell activation in the specific CTL clone.

The same assay as described supra was carried out, and Ca²⁺ mobilization following incubation with anti-HER2-Fab-HLA-A2/Flu conjugates, on SK-BR 3 cells, was studied.

Overall levels of mobilized Ca²⁺ observed following incubation with conjugate coated, SK-BR-3 cells, was comparable to that obtained with standard, anti-CD3 cross linking.

In contrast, the same anti-ErbB2 HA-A2 Flu conjugate in soluble form without the target cells, did not induce specific T cell activation. Thus, oligomerization of Fab-HLA-A2/Flu conjugates as a result of binding to cell surface to tumor antigens was shown to play an essential role on CTL activation.

### EXAMPLE 7

This example, and the examples which follow, describe the preparation and use of complexes which consist of Fab′ fragments, and streptavidin, conjugated to streptavidin/botin-MHC-peptide tetramers. Fab′ fragments from the antibodies described supra were used. The Fab′ fragments were conjugated to streptavidin by incubating a five molar excess of reduced Fab′ with streptavidin that had been derivatized with 4-8 mol of maleimede, for 16 hours, at 4° C., in 50 mM sodium acetate, 0.5 mM EDTA buffer, pH 7.0. The resulting Fab′-streptavidin conjugates were purified via FPLC. They eluted with an apparent molecular weight of 150-200 kilodaltons, suggesting 2-3 Fab′ molecules were coupled per streptavidin molecule. The streptavidin molecules conjugated to Fab′ fragments were used to assemble tetramers of biotinylated MHC/peptide complexes.

In brief, purified HLA-A*0201 heavy chain and β2M molecules were synthesized, using a commercially available prokaryotic expression system, using well known methodologies. The heavy chain was modified by deleting the transmembrane cytosolic tail, and the C-terminal addition of a sequence containing the BirA enzymatic biotinylation site. The heavy chain, β2M, and the peptide of SEQ ID NO:1 were refolded by dilution. The molecular weight of the desired product was 45 kilodaltons. Such products were isolated via FPLC, and then biotinylated in the presence of biotin, adenosine 5′-triphosphate, and Mg²⁺.

Following this, either Fab′ streptavidin conjugates, or free streptavidin was incubated, for 1 hour at 4° C., with the biotinylated, HLA-A*0201/peptide complexes, in a 1:4 molar ratio, and then concentrated to 1 mg/ml. See Altman, et al, Science 274:94-92 (1996); Romero et al, J. Exp. Med 188:1641-1650 (1998), incorporated by reference.

Three complexes, corresponding to the antibodies discussed supra, were made, i.e.:

anti-CEA-Fab-SA-A2/Flu

anti-HER2-Fab-SA-A2/Flu

anti-CD20-Fab-SA-A2/Flu

The conjugates of Fab, streptavidin, HLA-A*0201 and peptide eluted on FPLC at an apparent molecular weight of 350-400 kilodaltons, which suggests full tetramerization of the MHC on Fab′-streptavidin conjugates.

### EXAMPLE 8

These experiments describe the capacity of the Fab′-SA-MHC tetramers conjugates to coat HLA-A2 onto the four, HLA-A2 negative cell lines described supra. The cells were incubated with the conjugates in 20 μl of PBS-2% BSA, at a concentration of 100 μg/ml. After washing, the cells were incubated with FITC labelled, anti-HLA-A2 antibodies, as described supra, for an additional 30 minutes. The cells were washed, twice, in the same buffer, and analyzed immediately via FACS.

The colon carcinoma cell line LoVo, which is positive for CEA, gave a positive signal when preincubated with anti-CEA-Fab-SA-HLA-A2/Flu conjugate, but was negative when preincubated with anti-HER2-Fab-SA-HLA-A2/Flu. In similar fashion, SK-BR-3 and the Raji and Daudi lines gave a positive signal only when preincubated with either anti-HER2-Fab-SA-HLA-A2/Flu or anti-CD20-Fab-SA-HLA-A2/Flu

Following these experiments, titration assays were carried out, using concentrations of conjugate ranging from 3-200 μg/ml, under the same conditions.

The anti-ErbB-2-Fab-SA-HLA-A2 conjugate was the most potent, probably the result of the high affinity of the source antibody.

### EXAMPLE 9

These experiments describe the results of cell lysis assays carried out using the Fab′-SA-HLA-A2/Flu conjugates described, supra. The four cell lines described supra were used as targets. Samples of each cell line were incubated for 2 hours, at room temperature, with each of the different conjugates, at concentrations of 40 μg/ml. The cells in the samples were then washed three times, with PBS-BSA, and then labelled cells (1000 cell samples), were incubated with the CTL described supra, at effector:target cell ratios ranging from 0.1 to 30. Incubation took place in 200 μl of DMEM, 10% FCS, in V-bottom microwells, in the presence of 3 μg/ml human β2M. Chromium release was calculated as described supra.

As a negative control, ^{5l}Cr labelled target cells were preincubated with streptavidin-A2/Flu tetramers, without Fab, or an irrelevant Fab fragment, and tested with the same CTL.

Significant lysis was observed only when human cells were preincubated with conjugates containing the relevant anti-tumor marker antibody fragment. For example, anti-CEA-Fab-SA-HLA-A2/Flu induced lysis of CEA positive LoVo cells, while HLA-A2/Flu tetramer without Fab′, and anti-CEA-Fab-SA without HLA-A2 Flu, did not induce any detectable lysis. Similar results were obtained with the other lines.

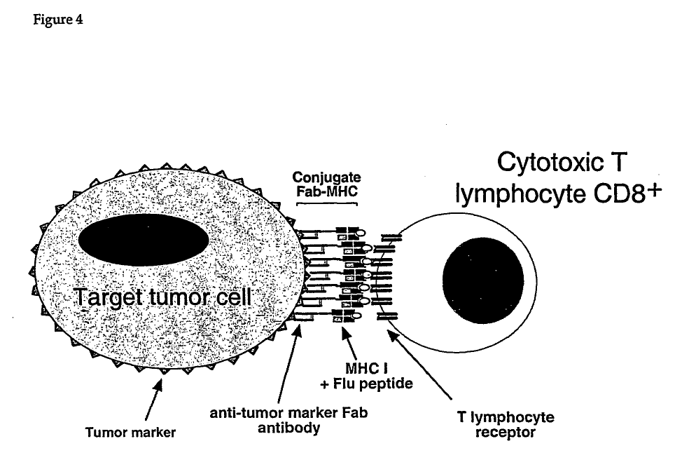

The mechanism by which the Fab′ antibody fragments conjugated to monomeric MHC/peptide conjugates induce efficient tumor target cell lysis is schematically described in FIG. 4. The key feature is that the bifunctional conjugates induce the binding and subsequent activation and cytolytic activity of CTLs, but only when they are oligomerized on tumor cells expressing a high density of tumor-associated antigen. Individual, soluble conjugates cannot bind and activate the specific CTL as shown in Example 6, due to the known low affinity of individual MHC complexes for the T cell receptor. See Altman et al. Science 274: 94-6 (1996) incorporated by reference.

This has important implications for clinical use of the described bifunctional conjugates, i.e., when intravenously injected, the above-described conjugates with monomeric MHC/peptide complexes will not activate specific T lymphocytes in the circulation. The bifunctional conjugates will be oligomerized on the cell surface, only when they have reached a tumor cell with high enough density of antigen or differentiation marker. Then, by cooperative binding, the oligomerized MHC/peptide complexes activate cytotoxic T lymphocytes and induce target cell lysis. Thus taking the old analogy of antibodies as guided missiles, here the missiles will be fired only when they reach their targets. Further, as a consequence of the absence of T cell activation by soluble bifunctional conjugates, such conjugates will be much less toxic than, for instance, the antibody-superantigen conjugates described by Dohlstein et al. Proc. Natl Acad. Sci US 88: 9287-9291 (1991), and Giantonio e al. J Clin. Oncol. 15: 1994-2007 (1997), incorporated by reference. Thus, the bifunctional conjugates described here can be injected in large enough amounts to target all accessible tumor cells. Still further, normal cells which may express small copy numbers of tumor-markers will not induce the CTL binding and lytic activity. This is significant, since numerous tumor markers or tumor associated antigens are know which are recognized by antibodies, and are abundant on cancer cells, but present at low densities on normal cells. Another practical advantage of conjugate or fusion protein made of single antibody fragment and monomeric MHC/peptide complex is that their relatively small size 95 kDa for instance for the conjugate described in Example 1 supra. This size is optimal for in vivo tumor targeting as shown for F(ab′)₂ fragments of 100 kDa from anti-CEA monoclonal antibodies in experimental and clinical studies. Buchegger et al. J Exp. Med. 158: 413-427 1983 and Delaloye et al. J Clin. Invet. 77: 301-311 1986 (for review, see Mach in Peckham M., Penedo, H. and Veronesi, U., Oxford Textbook of Oncology, Vol. 1, Oxford University press, pp-81-103 1995, incorporated by reference.

The foregoing disclosure sets forth the aspects of the invention, which relates to conjugates of formula

A—B—(C)n

wherein A is a specific binding protein, B is optional and, when present, is a binding partner to which both “A” and “(C)n” bind, “C” is a MHC combining a heavy chain of, e.g., a specific HLA molecule, a β₂M molecule, and a peptide, and “n” is a whole number, which preferably ranges from 1 to 10, most preferably 1, when A is a Fab′ fragment, and B is absent.

It is preferred that “A” is an antibody or a binding portion of an antibody, such as a Fab′ fragment or an F(ab′)₂ fragment or a single chain Fv fragment. The antibody or binding fragment is chosen so as to create a conjugate that binds specifically to an antigen, such as a cell surface tumor-associated or differentiation marker. The examples given supra, i.e., CD20, CEA, and HER2, are exemplary, but are by no means the only examples, of cell surface molecules to which the specific binding protein may be directed.

Whereas antibodies and binding fragments of antibodies are preferred, other binding proteins can be used. For example, the binding of receptor molecules and their specific ligand is well known. This specific binding arrangement can be exploited in preparation of the conjugates of the invention, such that “A” may be a ligand or receptor molecule, or a portion of such molecules known to be involved in receptor/ligand interaction. Exemplary of such interactions is that between epidermal growth factor (EGF) receptor and EGF and others are well known, and need not be repeated here.

As “B” is an optional part of the conjugates of the invention, it will be discussed infra. Attention now turns to “C”, which comprises an MHC/peptide complex. As was explained, supra, MHC molecules contain a heavy chain, a β2M molecule, and a peptide. Polymorphisms result in a wide variety of different types of MHC molecules, such as HLA-A1, A2, B27, Cw6, etc. These are all so-called “Class I” molecules. The art will also be familiar with “Class II” molecules, such as HLA-DR, and so forth. Any of these varieties of molecule may be used in the conjugates of the invention.

The MHCs also contain a peptide. As is well known in the art, the peptides which are a part of MHCs can, and do take various forms. Depending upon the nature of the HLA molecule, the nature of the peptide will change. There are various ways to choose the peptide which is used in the MHCs, such as using motif analysis, as described by Rammensee, et al, Immunogenetics 41:178-228 (1995); Ruppert, et al, Cell 74:929-937 (1993); Hunt, et al, Science 255:1261-1263 (1992); Falk, et al, Nature 351:290-296 (1991) all of which are incorporated by reference.

For inducing CTL lysis of cells, such as tumor cells, the conjugates may contain immunodominant, viral peptides, against which the patient has an active T cell memory repertoire. In the alternative, if the patient has an active T cell response against defined tumor rejection T cell antigens, the specific peptides involved in the response may be used. Another option is to use peptides known to be recognized by alloreactive T lymphocytes. A treatment protocol for a cancer patient using bifunctional antibody Fab-MHC/peptide conjugates in accordance with the invention, may include, e.g.:

A) HLA typing of the patient;

B) Analysis of the patient T cell repertoire against immunodominant common virus peptides, such as CMV, EBV or influenza viruses, restricted to his or her own MHC, as well as, in certain cases against the patient's own tumor rejection peptide antigens recognized by T lymphocytes;

C) Identification of the tumor markers, or tumor associated antigens or differentiation markers expressed more abundantly by the patient's tumor cells and recognized by available monoclonal antibodies;

D) Selection of the monoclonal antibodies according to the result of the analysis of point C and preparation of Fab′ fragment according to the invention;

E) Preparation of soluble MHC compatible with the patient HLA typing as described in supra, containing the MHC restricted most antigenic peptides selected according to analysis as above;

F) Synthesis of the Fab-HLA/peptide conjugate according to the invention;

G) Administer a booster of vaccination with the live virus from which the antigenic peptide was selected for making the conjugate, or a repeated course of active peptide immunotherapy with the selected tumor rejection antigenic peptide;

H) A few days after vaccination, boost or repeated peptide immunotherapy. The patient receives several intravenous injections of increasing doses of the bifunctional Fab-MHC/peptide conjugates;

I) Following in vivo targeting of the injected Fab-MHC conjugate on the tumor cells in vivo and the patient specific T lymphocytes lyse the MHC/antigenic peptide coated cancer cells, as if they were specifically injected by an antigenic virus.

When “B” is not present in the complexes, “A” and “C” may be prepared via the use of e.g., nucleic acid coding constructs which encode fusion polypeptides. Such techniques are well known, as is described, supra. One may also modify the elements “A” and “C” to connect them chemically, as was shown in the examples. One may add amino acid sequences such as those found in the Jun and Fos oncogenes, which then bind A and C via leucine zipper formation. Other alternatives are available, which the skilled artisan will note.

When “B” is used, this comprises a molecule or molecules which facilitates the linking of “A” and “C.” B can also comprise a specific binding pair of molecules, or a complex thereof, such as a complex of avidin or streptavidin or a chemically modified form of streptavidin or avidin, and anywhere from 1 to 4 biotin molecules. For example, B can be a bispecific antibody with one arm directed against a “Tag” epitope placed at the C terminus of A, and the other arm directed against another “Tag” epitope placed at the C terminus of C. The number of binding antibody fragments may vary. Preferably, from 1-5 are used. One may also use, e.g., a bifunctional antibody, or any other molecule or molecular complex to which “A” and “C” can both be joined such as an additional antibody, or binding fragment of an antibody. In particular, an additional antibody fragment which has the property of activating the T lymphocytes, such as anti-CD-28 antibody or a recombinant ligand, such as B7.1, B7.2, or IL-2 for a receptor that activates T lymphocytes, may be used. These additonal materials may be linked to a free cysteine, residue on the first Fab′ fragment from the Fab-MHC conjugate. The use of free cysteine on a bispecific antibody to synthesize trispecific antibodies is taught by Tutt, et al, J. Immunol 147:60-69 (1991), incorporated by reference. If fusion proteins are used, then a single cysteine residue allowing the coupling of the T lymphocyte activating, third molecule, can be introduced via, e.g., site specific mutation between the two partners of the fusion protein.

With respect to “n,” this will vary depending upon the nature of the other elements of the complex. When “B” is used, for example, there is potential for four biotin molecules, and each biotin molecule can be used to bind an MHC molecule. In such a case, “n” may range from 1 to 4, and is preferably 4. If “B” is an antibody, it can bind two molecules of “C,” and hence “n” will be “2.” The art is familiar with how to determine the number of elements in the conjugate.

The conjugates of binding partners and HLA/62 2 microglobulin/peptide may be labelled, using any of the labels known to the art, so as to monitor binding to target cells, to determine the number of bound conjugates, and to establish relationships between these values and the triggering of specific T lymphocytes. Examples of labels include enzymatic labels, such as alkaline phosphatase, metal particles, colored plastics made of synthetic materials, radioactive labels fluorescent labels, etc. Any of these may all be used.

The conjugates may be used, e.g., to identify or to isolate cytolytic T cells present in a sample, where these cells are specific for the HLA/β2 microglobulin/peptide complex. As the examples show, such cytolytic T cells bind to the immunocomplexes of the invention. In a preferred embodiment, the sample being tested is treated with a reactant which specifically binds to a cytolytic T lymphocyte of different phanalyne, wherein said label provides a detectable signal. The sample, including labelled CTLs, is then mixed with target cells coated with conjugates labeled with a fluorochrome. Labelled lymphocytes bind conjugate coated target cells, forming cell clusters which can be separated, preferably by FACS, or by any of the standard, well known approaches to cell separation, such as magnetic cell sorting or density gradient centrifugation. Another separation method can be incubation of the T lymphocyte samples with immobilized target cells coated with different Fab′-MHC/peptide complexes. The peptide used may be chosen by the skilled artisan, depending upon the nature of the specific MHC system under consideration.

Additionally, the method can be used to monitor the status of lymphocyte reactivity against tumors, following administration of a particular therapeutic agent, such as a vaccine. Functional T lymphocyte activation and cytolytic tests can be performed on patient T lymphocytes incubated with target cells coated with bifunctional Fab′-MHC/peptide conjugates. The use of, e.g., Daudi cells devoid of MHC Class I molecules as a target, permits the artisan to determine, e.g., the number of anti-CD20 Fab′-MHC Class I with different peptides, such as TRAs, necessary for inducing patient T lymphocyte activation and cytotoxicity. Further, the methodology can be used to identify cytolytic T cell precursors.

Also apart of the invention is the use of conjugates as described, in conjunction with other steps, to yield populations of T cells with desired features, such as specificity and phenotype. These include distinct cell surface phenotypes associated with antigen experienced, or memory cells, or naive cells, and so forth. Such populations can be cultured, in the presence of either bifunctional Fab′-MHC/peptide conjugate coated target cells, or free peptides in target cells to determine to what extent the deletion of MHC molecules are responsible for the lace of reactivity of T lymphocytes for CTLs. This culturing can be carried out with a mitagen such as phytohemagluttinin, e.g., without peptides, for comparison.

The invention also involves methods for obtaining desired T cells via in vitro or by in vivo recruitment, using the same type of bifunctional conjugates in which the antibody or binding protein is directed against a surface marker expressed by “antigen presenting cells.” In this case, one can predict that, by a similar oligomerization of MHC on target cells, as described in FIGS. 1 and 4 supra, the targeted antigen presenting cells will stimulate the activation and proliferation of functionally naive T lymphocytes specific for the peptide associated with the bifunctional conjugates. This presentation of selected peptide can be used to improve vaccinations approaches.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

We Claim:

1. A conjugate or fusion protein of formula:
A—B—(C)n
wherein A is a protein or polypeptide which binds specifically to a target cell surface, B is optionally present, and comprises at least one molecule which binds to both A and (C)n, C is an MHC/peptide complex, and n is a whole number ranging from 1 to 10.

2. The conjugate of claim 1, wherein B is absent, and n is 1.

3. The conjugate of claim 2, wherein A is an antibody or a binding fragment of an antibody.

4. The conjugate of claim 4, wherein A is an Fab′ fragment of an antibody.

5. The conjugate of claim 2, wherein A is a single chain antibody.

6. The conjugate of claim 4, wherein C is a single chain MHC complex.

7. The conjugate of claim 5, wherein C is a single chain MHC complex.

8. The conjugate of claim 1, wherein A is an antibody, a ligand which binds to an antigen, or a ligand which binds to a differentiation marker overexpressed in tumor cells.

9. The conjugate of claim 1, wherein B is present.

10. The conjugate of claim 5, wherein B comprises a streptavidin or avidin molecule, and from 1 to 4 biotinylated MHC molecules.

11. The conjugate of claim 10, wherein B comprises a streptavidin molecule and 4 biotin molecules.

12. The conjugate of claim 11, wherein A comprises an antibody binding fragment.

13. The conjugate of claim 11, wherein A is an Fab′ fragment.

14. The conjugate of claim 1, wherein said MHC molecule comprises a tumor rejection antigen.

15. The conjugate of claim 1, wherein said MHC molecule comprises an antigenic, viral peptide.

16. The conjugate of claim 2, wherein A is a ligand which binds to a receptor.


SEQUENCE LISTING (1 sequence)

SEQ ID NO: 1 - protein, 9 aa, H. influenzae
GILGFVFTL